(19) United States
(12) Patent Application Publication
(10) Pub. No.: US 2009/0157044 A1
(43) Pub. Date: Jun. 18, 2009

(54) Epidural administration systems

(75) Inventors: Keith Liyanagama, New South Wales (AU); Terry Thyssen, New South Wales (AU)

Correspondence Address: FACTOR & LAKE, LTD, 1327 W. WASHINGTON BLVD., SUITE 5G/H, CHICAGO, IL, 60607, US

(73) Assignee: NOVODURAL PTY LTD

(21) Appl. No.: 11/990,952

(22) PCT Filed: Aug. 25, 2006
(86) PCT No.: PCT/AU2006/001243

§ 371 Date: Feb. 25, 2008

(30) Foreign Application Priority Data
Aug. 26, 2005 (AU) 2005904666

(51) Int. Cl. A61M 19/00; A61M 5/31; A61M 29/00; A61M 5/00; A61M 25/00; A61M 5/32; A61M 5/315

(52) U.S. Cl. 604/512; 604/187; 604/104; 604/264; 604/523; 604/272; 604/218

(57) ABSTRACT

The invention relates to improved equipment for the administrations of epidural anaesthetic and to novel methods of administration using the same. Improvements include a needle configured for reduced incidence of post dural puncture headaches, an improved sensitivity loss of resistance syringe and a novel cannula which in one form readily facilitates combined spinal epidural anaesthesia (CSE). Also includes is a catheter having a pre-assembled injection hub and connection means to optionally retain a cannula that is withdrawn over the catheter.

### FIELD OF THE INVENTION

The present invention relates to improved equipment for the administration of epidural anaesthetic and to novel methods of administration using the same. While the improvements relate primarily to epidural anaesthesia, preferred forms of the inventions as described readily facilitate combined spinal epidural anaesthesia (CSE).

### BACKGROUND OF THE INVENTION

The most commonly used existing equipment for administering epidural anaesthesia has several drawbacks both in terms of ease of use by the anaesthetist and the incidence level and severity of error related side effects such as post dural puncture headaches and the like.

In this regard, the most widely used equipment for placement of epidural catheters uses a relatively large 16-18 gauge hollow needle having a slant cut piercing point which is known as a Touhy needle. This needle is attached to a syringe (typically a 10 ml syringe) filled with either air or saline. Transition of the needle tip from the over lying external tissue to the underlying epidural space is identified by the plunger of the syringe readily moving forward as the needle enters the epidural space. This is known as the “loss of resistance” technique. The syringe is then removed and an epidural catheter passed through the Touhy needle which is then removed.

However, if the epidural space, which has a depth of only approximately 2-3 mm, is not identified accurately and immediately by the operator, there is a high risk the sharp slant cut point of the needle may puncture the dura lying therebelow. The risk of a headache following dural puncture with a 16 gauge Touhy needle is up to 75%.

It is an object of the present invention to provide an improved apparatus and method for administration of epidural anaesthesia, which overcomes or ameliorates one or more of the above discussed disadvantages of the prior art or which at least offers a useful alternative.

### DISCLOSURE OF THE INVENTION

According to a first aspect of the invention there is provided an improved epidural needle having:

a lumen having a first axis parallel to said needle that is sized to receive a guide wire therethrough;

a tissue piercing point located at a distal end; and

a lumen opening disposed at a location at or closely adjacent said tissue piercing point, said opening being configured to deflect a guide wire exiting from said opening in a direction away from said first axis;

wherein said needle gauge is 20 or finer.

Desirably, the needle gauge is in the range of 20-25 with a corresponding nominal inside diameter of 0.65-0.3 mm, preferably with a corresponding nominal outside diameter of 0.90-0.5 mm. Similarly, the preferred wire gauge is 24-31 which correlates to a nominal outside diameter range of 0.56-0.27 mm.

Preferably, the needle gauge is 22 or finer. In the preferred form, the needle tissue piercing point is of a pencil point configuration.

According to a second aspect of the invention there is provided a cannula for introducing an epidural catheter, said cannula having a generally straight tubular portion terminating in a discharge section having a discharge opening, said discharge section and discharge opening being configured such that, in use, a catheter passed therethrough is caused to deflect in a direction away from the axial extent of said generally straight tubular portion.

In one preferred form the cannula gauge is 16-20 with a corresponding nominal inside diameter of 1.32-0.65 mm, preferably with a corresponding nominal outside diameter of 1.65-0.90 mm.

Desirably, in use the discharge section of said cannula is bent away from the axis of said straight tubular portion.

In the preferred form, the cannula is formed from a resilient material that has some form of “shape memory” whereby the cannula discharge section can be straightened for insertion through or over a dilator and guide wire and then regain its bent or deflecting configuration when the dilator is removed.

Preferably the cannula is made from a polymeric material and has a rounded distal end.

In a preferred form the cannula may be pre-assembled with a suitably sized internal or external dilator. In some preferred embodiments the dilator gauge is 17-19 which corresponds to having a nominal inside diameter of 1.17-0.8 mm, preferably with a corresponding nominal outside diameter of 1.17-0.8 mm.

In accordance with a third aspect of the invention there is provided a cannula for introducing an epidural catheter which includes an integrally formed co-extensive needle guide. Preferably the cannula also includes the features of the second aspect of the invention listed above.

According to a fourth aspect of the invention there is provided a catheter having a connection for attachment of a bacterial filter and for providing an injection hub, said connection means also including retention means to secure a cannula thereto after withdrawal once the catheter is in place.

The preferred catheter gauge is in the range of 18-22 with a corresponding nominal inside diameter of 0.96-0.46 mm and corresponding nominal outside diameter of 1.27 to 0.71 mm.

In a preferred form the proximal end of the cannula is adapted to screw onto or into said retention means on the connection means at the proximal end of the catheter or connect thereto by some other means. Preferably interconnection of the cannula and retention means is via a Luer lock type connector.

Alternatively a splittable introducing cannula is employed.

Preferably, the catheter is pre-assembled with a bacterial filter and injection hub via the connection means.

According to a fifth aspect of the invention there is provided an improved sensitivity syringe for use in loss of resistance needle placement techniques, said syringe including:

an external generally tubular housing of volume A;

an inner syringe assembly having a barrel of volume B (where A is greater than B) with a plunger rod and piston assembly reciprocally axially movable therein and a discharge opening at its distal end;

said barrel of said internal syringe assembly being located generally co-extensively within said external tubular housing.

In one preferred form the housing terminates in a generally tubular discharge tip and the internal syringe discharge opening is sealingly connected to the discharge tip. In an alternative form the discharge tip of the syringe is formed as part of the inner syringe assembly.

Preferably, the syringe further includes indicator means connected with said plunger rod so as to enhance the detection of movement of said internal plunger rod and piston assembly.

In the preferred form the external tubular housing is sized to be similar to that of a conventional 10 ml syringe and the internal syringe assembly has a significantly smaller volume such as, for example, 2 ml.

In one form the indicator means includes an indicator tab or ring that is rigidly connected via an extension member to a location at or adjacent an operator end of the plunger rod, movement of which can be viewed against markings provided on the external housing. In the preferred form the housing is formed from a translucent or transparent material and the indicator tab or ring located within the space between the internal syringe assembly and the external tubular housing so that movement is clearly visible through the translucent housing which may include markings to enhance recognition of plunger movement. Alternatively, the indicator tab or ring can be located on the outside of the external tubular housing.

In other forms the indicator means may comprise some form of indicator light, audible sound or equivalent which may optionally be connected to some form of pressure, movement or flow sensor operatively connected to the internal syringe assembly.

In accordance with a sixth aspect of the invention there is provided a method of administering epidural anaesthesia comprising the steps of:

inserting an epidural needle in accordance with the first aspect of the invention into the skin over the epidural space;

using a “loss of resistance” syringe to guide the epidural needle through the external tissue until the tip of the needle including the lumen opening is located within the epidural space;

inserting a guide wire through the needle and into the epidural space;

removing the needle;

passing a dilator over said guide wire to the depth of the epidural space;

removing the dilator;

inserting an introducing cannula via said guide wire;

removing the guide wire; and

inserting the epidural catheter through the cannula and into the epidural space.

In accordance with a seventh aspect of the invention there is provided a method of administering epidural anaesthesia comprising the steps of:

inserting an epidural needle in accordance with the first aspect of the invention into the skin over the epidural space;

using a “loss of resistance” syringe to guide the epidural needle through the external tissue until the tip of the needle including the lumen opening is located within the epidural space;

noting the depth of the epidural space;

inserting a guide wire through the needle and into the epidural space;

removing the needle;

introducing a cannula that is preloaded onto the outside of a dilator by passing the dilator and cannula assembly over said guide wire until the cannula reaches the noted depth of the epidural space;

removing the dilator and guide wire; and

inserting the epidural catheter through the cannula and into the epidural space.

In one form the method of either the sixth or seventh aspect then includes the step of removing or at least retracting the cannula prior to introducing epidural anaesthesia through the catheter.

In the preferred method a catheter according to the fourth aspect is used which has a pre-assembled bacterial filter and injection hub. Preferably the cannula is retracted after the catheter is in place and is secured to the connection means adjacent the bacterial filter and injection hub. Alternatively, a splittable cannula is used which can be removed completely.

In another form, the method is varied to enable combined spinal epidural anaesthesia (CSE). In this method, an introducing cannula in accordance with a third aspect is used, the method then including the additional steps of:

inserting a spinal needle through the needle guide of the cannula and into the dura (after the epidural catheter has been placed within the cannula);

administering a spinal anaesthesia through said spinal needle and removing same; and

removing or withdrawing the epidural cannula leaving only the epidural catheter in place.

According to an eighth aspect of the invention there is provided an epidural anaesthesia kit including:

an improved epidural needle in accordance with the first aspect of the invention;

a guide wire sized for use with said epidural needle;

a loss of resistance syringe for connection to said needle;

a dilator configured for insertion over said guide wire;

a cannula configured for insertion via said dilator; and

an epidural catheter.

Preferably, the “loss of resistance” syringe is a syringe in accordance with the fifth aspect of the invention. Similarly, in the preferred form, the cannula is a cannula in accordance with either the second or third aspects of the invention.

Optionally, the catheter includes a pre-assembled injection hub and bacterial filter. In one form, this assembly may also include a pre-attached cannula which is adapted for releasable attachment at or adjacent a proximal end of the catheter for retention in transport and/or after retraction once the catheter is placed.

In one form specifically configured for combined spinal epidural anaesthesia (CSE), the kit preferably includes the cannula of the third aspect of the invention and further optionally includes a spinal needle sized for use with the same.

### BRIEF DESCRIPTION OF THE DRAWINGS

Preferred forms of the various aspects of the invention will now be described, by way of example only, with reference to the accompanying drawings in which:

FIG. 1 is a longitudinal sectional side view of a first embodiment improved epidural needle in accordance with the first aspect of the invention in use;

FIG. 2 is a perspective view showing detail of the needle and guide wire of FIG. 1;

FIG. 3 is a longitudinal sectional view showing a dilator-positioned-over the guide wire of the previous Figures;

FIG. 4 is a sectional side view showing the guide wire removed and an epidural catheter inserted through a first embodiment cannula previously introduced via the guide wire;

FIG. 5 is an enlarged end view of the cannula of FIG. 4 showing the tip of the cannula in the straight “insertion” mode as when passing over the guide wire through the tissue opening and the bent or directed “placement” mode when the distal end is in the epidural cavity;

FIG. 6 is a series of views showing a second embodiment cannula including a spinal needle guide configured for use in combined spinal epidural anaesthesia (CSE);

FIG. 7 is a longitudinal sectional view showing the epidural catheter in place and a spinal needle inserted through the needle guide of the cannula shown in FIG. 6;

FIG. 8 is a side view illustrating the cannula in a retracted position with only the epidural catheter remaining;

FIG. 9 is a sectional side view of a first embodiment improved sensitivity “loss of resistance syringe” in accordance with the fifth aspect of the invention.

FIG. 10 is a sectional side view of an optional transducer assembly for use with an alternative syringe in “loss of resistance” needle placement techniques; and

FIG. 11 is another alternative syringe assembly suitable for use in the method or kit of the invention.

### PREFERRED EMBODIMENTS OF THE INVENTION

Referring to the drawings, there is described improved methods and apparatus components for use in the administration of epidural anaesthesia. The improved apparatus components include: a novel fine gauge epidural needle, improved catheter introducing cannulas, a novel catheter which may form part of a pre-connected sub-assembly; and an improved sensitivity syringe for use in loss of resistance needle placement techniques which is suited to proposed new methods of administration of epidural anaesthetic as described hereafter.

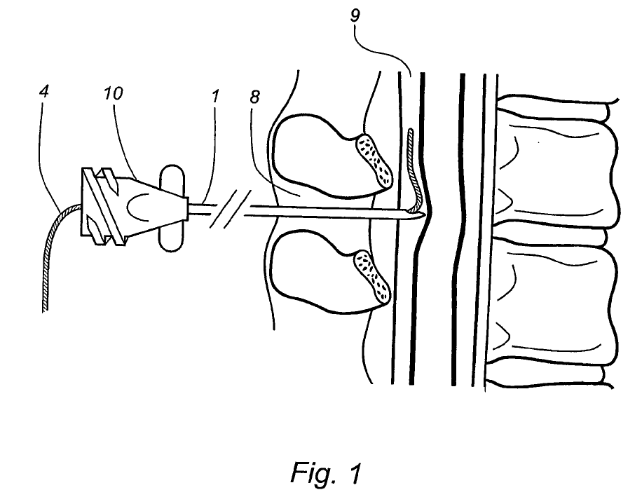

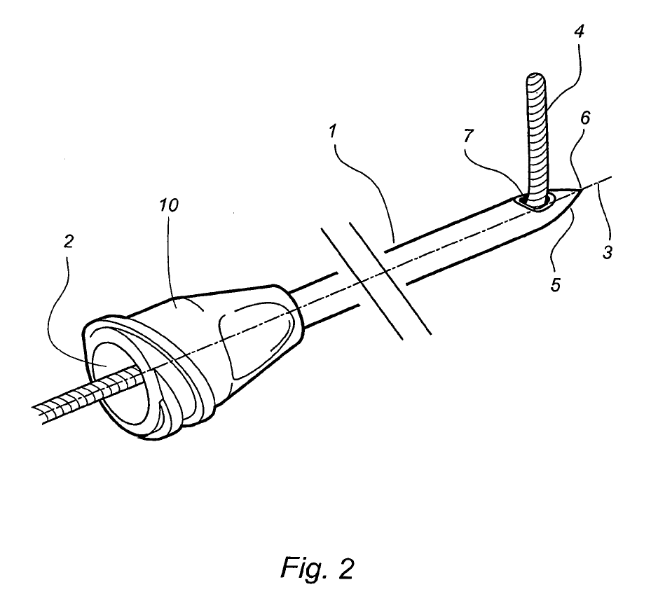

Referring firstly to FIGS. 1 and 2 there is shown an improved epidural needle 1 having a lumen 2 having a first axis 3 that is sized to receive a guide wire 4 therethrough. Desirably, the needle gauge is in the range of 20-25 with a corresponding nominal inside diameter of 0.65-0.3 mm, preferably with a corresponding nominal outside diameter of 0.90-0.5 mm. Similarly, the preferred wire gauge is 24-31 which correlates to a nominal outside diameter range of 0.56-0.27 mm.

Located at the distal end 5 of the needle is a tissue piercing point 6 adjacent which is a lumen opening 7 which is configured to deflect a guide wire exiting from the opening in a direction away from the first axis. The external gauge of the needle is greater than 20 and more preferably is 22 or finer. In the preferred form illustrated, the needle tissue piercing point 6 is of a pencil point configuration. However, other point configurations could be used.

As can be seen from FIG. 1, the needle has been inserted through the tissue 8 overlying the epidural space shown at 9. The proximal end of the needle preferably terminates in a standard Luer lock fitting 10, or other suitable connection means, for releasable attachment to a “loss of resistance” syringe as described in more detail below.

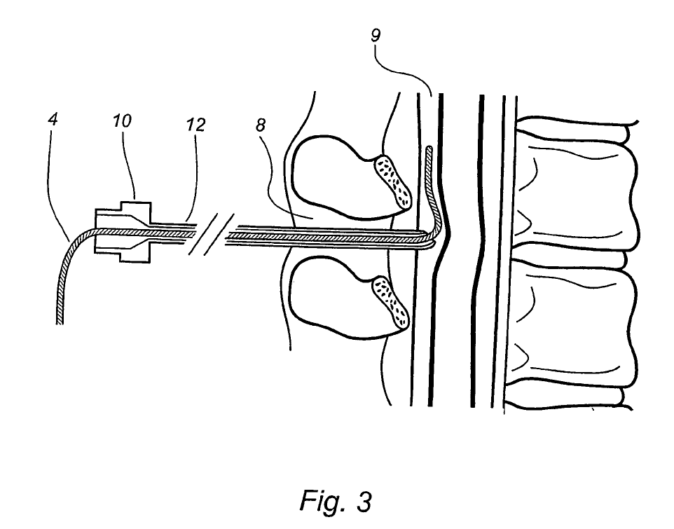

Referring next to FIG. 3, there is shown a first embodiment dilator 12 which has been inserted over the guide wire 4 after the needle 1 has been removed.

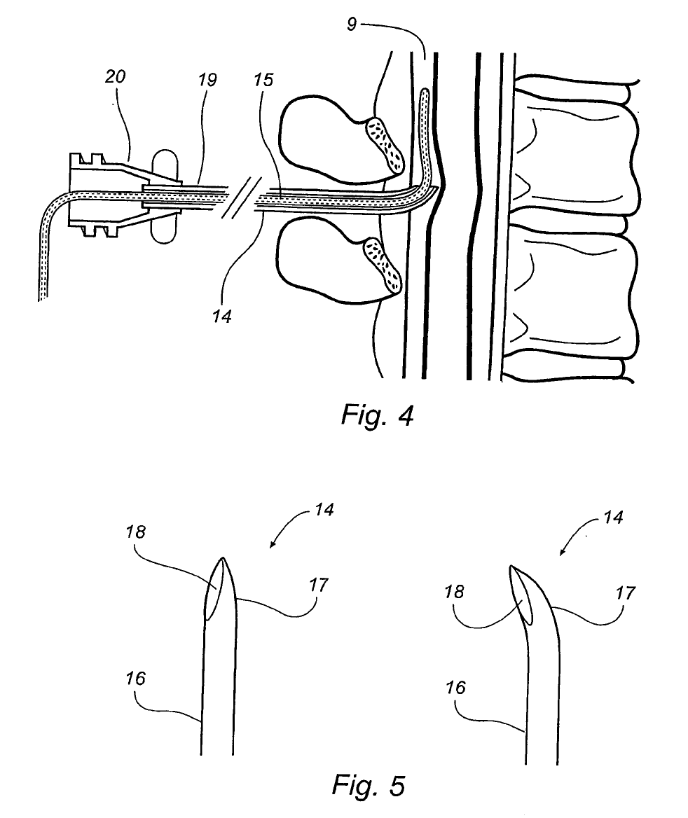

FIGS. 4 and 5 illustrate a first embodiment cannula 14. In one preferred form the cannula gauge is 16-20 with a corresponding nominal inside diameter of 1.32-0.65 mm, preferably with a corresponding nominal outside diameter of 1.65-0.90 mm.

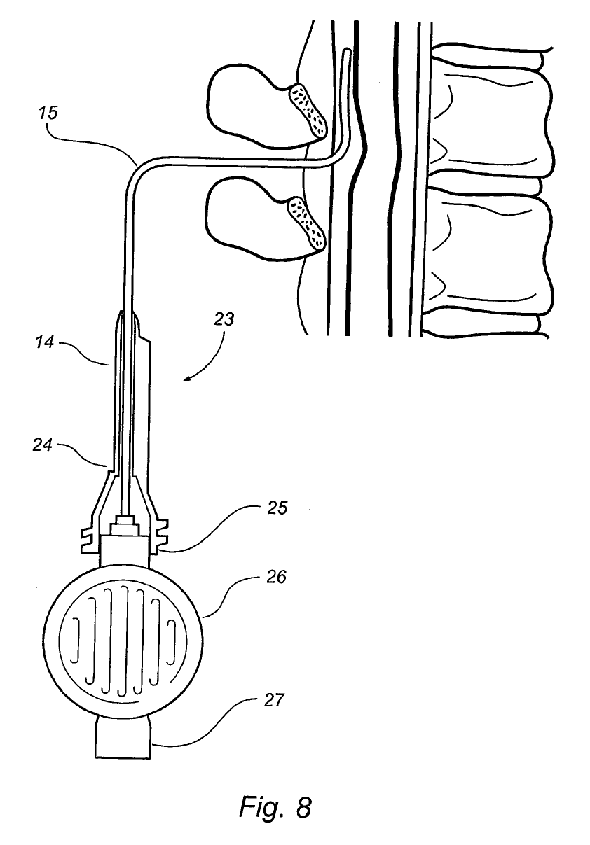

As shown in FIG. 4, the cannula directs an epidural catheter 15 into the epidural cavity 9. The cannula has a generally straight tubular portion 16 which terminates in a discharge section 17 having a discharge opening 18 at its distal end. The proximal end 19 of the cannula includes a fitting 20, which again maybe a Luer lock type connector or screw fitting, which may be used for connection of the cannula to a hub portion of a catheter assembly as will be described in more detail in reference to FIG. 8.

Preferably, the discharge section of the cannula 14 is bent and is formed from a resilient material that has some form of “shape memory”. In this manner the cannula discharge section 17 can be straightened for insertion through or over a dilator and guide wire and then regain its bent or deflecting configuration when the dilator is removed so that in situ the discharge opening 18 is directed into and along the epidural space 9.

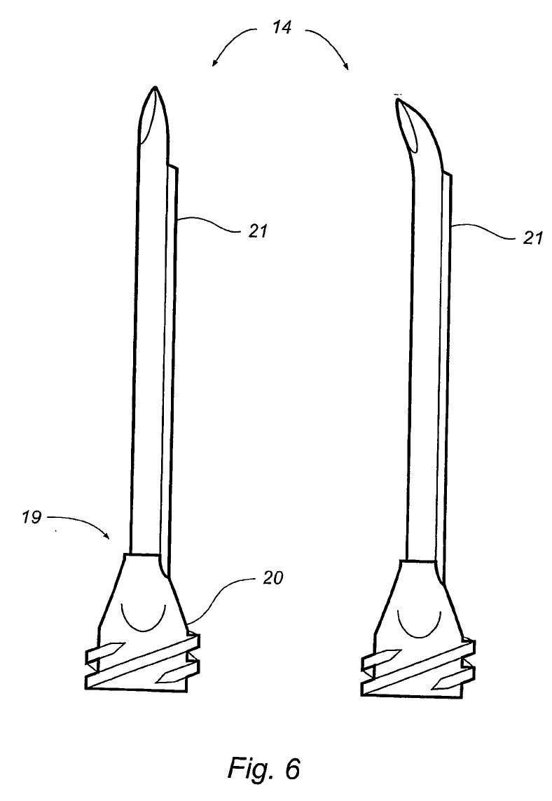

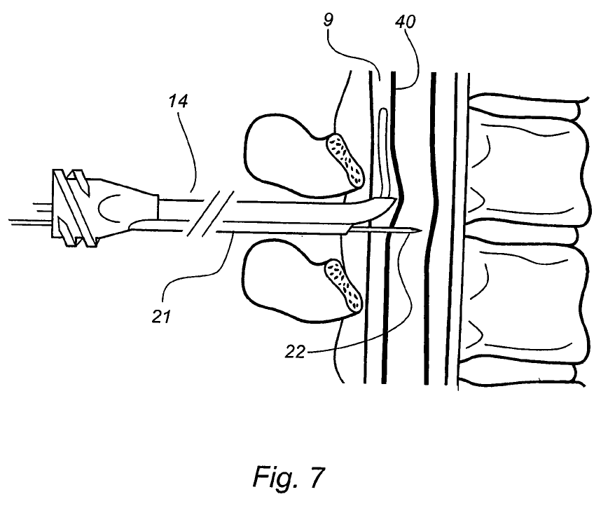

In one preferred form of the invention as shown in FIGS. 6 and 7, the cannula includes an integrally formed co-extensive needle guide 21 adapted for guiding a spinal needle 22 so as to readily facilitate combined spinal epidural anaesthesia (CSE).

Referring next to FIG. 8 there is shown an improved catheter arrangement 23 which includes a catheter 15 which terminates at its proximal end 24 in a connector port 25. This port is adapted for attachment of a bacterial filter 26 as shown which also provides an injection hub 27. Desirably, the catheter arrangement also includes retention means to secure a cannula thereto after withdrawal once the epidural catheter is in place. In one form, the retention means forms part of the assembly comprising the connection port 25.

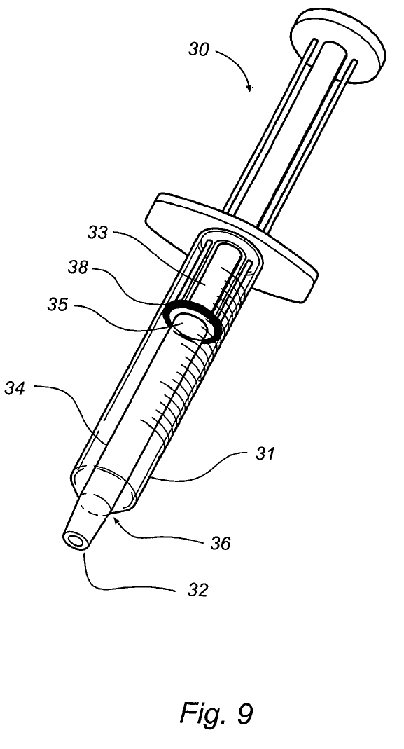

Referring finally to FIG. 9, there is shown a first embodiment improved sensitivity syringe 30 for use in loss of resistance needle placement techniques. The syringe 30 includes an external tubular housing 31 of volume A. Located generally co-extensively within the external tubular housing 31 is an inner syringe assembly 33 having a barrel 34 of volume B (where A is>than B). A reciprocally movable plunger rod and piston assembly 35 is provided in the barrel 34. The barrel has a discharge opening 36 at its distal end which is connected to a discharge end 32 of the external housing 31. In the preferred form illustrated, the housing 31 terminates in a discharge tip and the internal syringe discharge opening is sealingly connected to that discharge tip. In other forms the discharge tip of the syringe is formed as part of the inner syringe assembly.

The syringe 30 also includes indicator means 38 connected with the plunger rod assembly 35 so as to enhance the visibility of movement of the internal syringe assembly. In order to ensure the internally located indicator means 38 is clearly viewable, the external tubular housing is preferably formed from a translucent or transparent material and/or may include some form of window means therein. In another embodiment the indicator means is provided external to the external housing by means of an offset parallel arm mechanism or the like. In other variations the indicator means may comprise some form of indicator light or equivalent which may optionally be connected to some form of pressure sensor, motion sensor, flow sensor or proximity sensor operatively connected to the internal syringe assembly.

The external tubular housing 31 is sized to be similar to that of a conventional 10 ml syringe and the internal syringe assembly has a significantly smaller volume such as, for example, 2 mls. In this manner the movement of the plunger is magnified 300%.

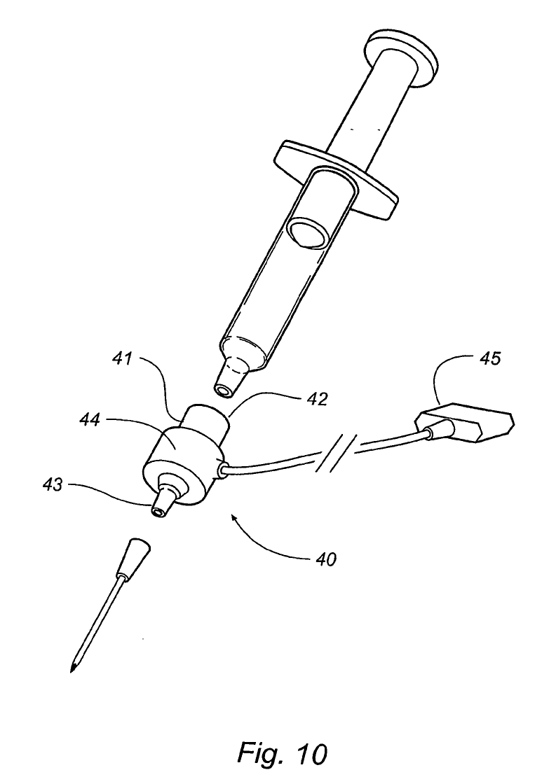

An enhancement to the improved sensitivity syringe of FIG. 9 which could also be used with conventional syringe assemblies is the use of a transducer assembly 40 as shown in FIG. 10. This assembly includes a housing 41 having a passage therethrough which has a first connection port 42 for connection to a syringe and a second port 43 for connection to a hollow needle. Within the housing is a transducer which includes a sensor 44 operatively connected with the through passage that is responsive to volume or pressure variations with the syringe indicative of changes in resistance felt as a result of tissue density variations at the needle tip. The device also includes an output connector 45 to connect the transducer to an indicator device (not shown) to register a predetermined change in resistance or volume. The indicator device may include a light signal and/or audio signal or any other suitable indicator means. In another variation this indicator may be formed to be part of the transducer assembly. Alternatively the transducer can be interconnected to an Analogue to Digital Converter for translation of the signal into the digital domain and display on a computer display screen.

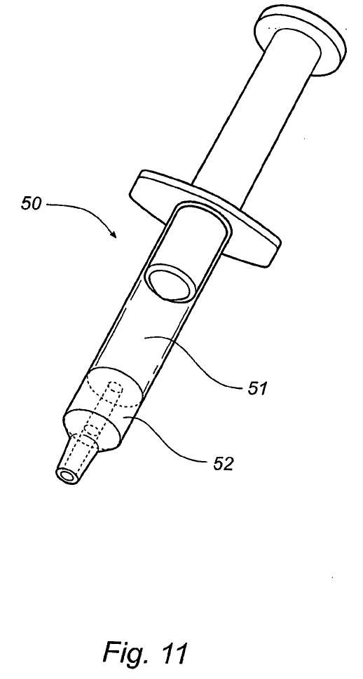

The concept of some form of sensor and/or transducer assembly can also be applied directly to the syringe as shown in FIG. 11. In this embodiment the syringe 50 is of a conventional design externally, but includes within the piston chamber 51 a transducer and indicator device 52. This device includes suitable sensors that are responsive to pressure and/or volume changes in the chamber and also includes suitable output or indicator means such as a light.

Improved methods of administering epidural anaesthesia will now be described with reference to the apparatus components described above.

In a first improved method, the epidural needle 1 is inserted into the skin over the epidural space 9. A “loss of resistance” syringe, preferably of the kind described in reference to one of FIGS. 9, 10 or 11 is attached to the proximal end of the needle 1 using a Luer lock connector 10 or the like. The attached syringe is filled with either air or saline. As the needle is directed inwardly toward the dura opposite the epidural space, the tissue effectively blocks the needle opening thereby inhibiting movement of the plunger. As the needle 1 enters the epidural space 9, the needle opening clears and the plunger of the syringe moves forwards due to the loss of resistance which translates to a loss of volume in the syringe. This movement is visually enhanced by the indicator means 38 of the syringe of FIG. 9, (or the indicator means connected via output connector 45 to sensor 44 of FIG. 10 or sensor 52 in FIG. 11) and signifies that the needle tip has entered the epidural space and the syringe is then removed. The guide wire 4 is then inserted through the needle 1, the curved lumen opening 7 guiding the wire 4 away from the axis of the needle and into the epidural space. Preferably, using markings provided on the external surface of the needle 1, the depth of the epidural space is noted and then the needle 1 is removed.

A dilator 12 is then inserted over the guide wire 4 as shown in FIG. 3 to the predetermined depth of the epidural space. This serves to widen the opening and thereby better facilitate subsequent introduction of the cannula 14 and catheter 15. The dilator is then removed and the introducing cannula inserted over the guide wire. Once the cannula is in place with its discharge opening 18 carefully located in the epidural space 9, the guide wire 4 is removed and the catheter inserted as shown in FIG. 4. In one variation to this method, the cannula 14 is pre-loaded with a dilator and this combined assembly is fed over the guide wire until the cannula reaches the noted depth of the epidural space, after which again the dilator and guide wire are removed. The epidural catheter 15 is then inserted through the cannula and into the epidural space as described above.

In one particularly preferred form of the invention, the catheter 15 forms part of a catheter sub-assembly 23 comprising the catheter 15 having at its proximal end 24 connection port 25. A bacterial filter 26 is attached to the connection 25.

In one preferred form, the cannula 14 has a fitting 20 at its proximal end 19 that enables the cannula to be withdrawn and secured at or adjacent the connection port 25 of the catheter. This eliminates any disconnection risks and also allows the use of the catheter assembly having a pre-connected bacterial filter and injection hub. Use of such a catheter and bacterial filter sub-assembly would also be enabled by the use of a cannula 14 that is longitudinally split to allow removal transverse to the axis of the catheter. All of these variations offer substantial advantages in that at least some of the components can be pre-assembled in the factory, minimising infection and disconnection risks and eliminating need for assembly at the bedside as is currently required because of the need to completely remove the needle over the catheter.

In another form, the method is varied to enable combined spinal epidural anaesthesia (CSE) as shown for example in FIG. 7. This method uses the preferred cannula of the third aspect of the invention which includes an integrally formed co-extensive needle guide 21. The method is essentially the same as the method described above, but includes the additional steps of inserting a spinal needle 22 through the needle guide 21 of the cannula and into the dura 40 after the epidural catheter 15 has been placed within the cannula. Spinal anaesthesia is then administered through the spinal needle and the needle is removed. The epidural cannula is then removed or withdrawn and retained on the catheter leaving only the epidural catheter in place.

The advantages of the various aspects of the invention are numerous. For example, the use of the improved epidural needle, which is a different shape and of a significantly finer gauge than the Touhy needles of the prior art, substantially reduces the risk of a headache following inadvertent dural puncture.

Furthermore, the actual risk of dural puncture is also substantially reduced by use of the improved sensitivity syringe of the fifth aspect of the invention which is designed to more accurately communicate passage of the needle into the epidural space compared with the state of the art. By maintaining the external dimensions of the syringe the device remains easy to hold and is familiar to operators that regularly use the prior art syringes. However, the loss of resistance or loss volume is effectively magnified and translates to a larger indicator movement by use of the smaller volume internal syringe chamber. It will be clearly apparent that while this novel syringe arrangement is particularly suited for use in the administration of epidural anaesthesia as described herein, it is also suitable for use in other procedures where a “loss of resistance” syringe is typically employed.

Additionally, by introducing the catheter via a cannula, rather than through a needle as was done in the prior art, accurate combined spinal epidural anaesthesia is readily facilitated with the specially configured embodiment of the cannula shown in FIGS. 6 and 7. The cannula may optionally include the curved “shape memory” tip of the preferred embodiment, as the spinal needle guide 22 does not extend the fill length of the cannula.

The current art involves locating the epidural space in the standard fashion with a conventional loss of resistance syringe and a Touhy needle. A spinal needle is then passed via the Touhy needle until it pierces the dura, following which clear cerebro spinal fluid (CSF) is seen. Anaesthetic is then injected intrathecally (into the CSF), then the spinal needle removed. Finally an epidural catheter is passed into the epidural space before the Touhy needle is removed.

This prior art technique has several limitations, the chief being that the spinal anaesthetic begins to work immediately. If there is then a technical problem with the placing of the epidural catheter such as being unable to feed it or blood flowing back within the catheter (suggesting the catheter might be lying within a vein), then in the context of a caesarean section the epidural maybe abandoned and the case proceed under spinal anaesthetic alone, without the provision for intra-operative supplementation or post-operative analgesia.

By contrast, the proposed technique of the present invention involves placing a cannula within the epidural space as previously described, then feeding the epidural catheter and confirming that this is in order. Only then, after the clinician is happy that the epidural catheter is satisfactorily placed, do they commence the spinal anaesthetic.

Finally, as alluded to above, the epidural cannula need not have a sharp tip (as does the prior art Touhy needle) because it is fed with an introducer over a wire, and therefore does not need to be removed from the assembly when the insertion is complete. As described, the cannula can simply be slid back to the proximal end of the catheter assembly 23 and held there with a simple threaded arrangement or Luer lock connector or the like. This allows the epidural catheter, bacterial filter and connection for syringes/infusions to be pre-assembled in the factory. Currently, the operator must do this after scrubbing and before commencing the procedure. The assembly and method of the present invention thereby greatly reduces the risks of contamination, both from inadvertent operator contamination at assembly, as well as secondary contamination relating to disconnection of the components, a particular problem in labouring maternity patients who change position frequently in response to their labour.

Although the invention has been described in detail with reference to specific examples, it will be appreciated that the invention may be embodied in many other forms.

1. A method of administering epidural anaesthesia comprising the steps of:
inserting an epidural needle of a gauge of 20 or finer into the skin over the epidural space, the epidural needle having a lumen having a first axis parallel to said needle that is sized to receive a guide wire therethrough, a tissue piercing point located at the distal end, and a lumen opening disposed at a location at or closely adjacent the tissue piercing point, the opening being configured to deflect a guide wire exiting from the opening in a direction away from said first axis;
using a “loss of resistance” syringe to guide the epidural needle through the external tissue until the tip of the needle including the lumen opening is located within the epidural space;
inserting a guide wire through the needle and into the epidural space;
removing the needle;
passing a dilator over said guide wire to the depth of the epidural space;
removing the dilator;
inserting an introducing cannula via said guide wire;
removing the guide wire; and
inserting the epidural catheter through the cannula and into the epidural space.

2. (canceled)

3. The method of claim 1 including the further step of removing or at least retracting the cannula prior to introducing epidural anaesthesia through the catheter.

4. The method of claim 1 wherein the catheter has a pre-assembled bacterial filter and injection hub including a cannula connection means and the method includes the step of retracting the cannula after the catheter is in place and securing it to the connection means adjacent the bacterial filter and the injection hub.

5. The method of claim 1 wherein a split cannula is used and the method includes the step of removing the cannula after the catheter is in place.

6. The method of administering epidural anaesthesia of claim 1 wherein the cannula includes a separate integrally formed at least partially co-extensive needle guide, the method then including the additional steps of:
inserting a spinal needle through the needle guide of the cannula and into the dura;
administering a spinal anaesthesia through said spinal needle and removing same; and
removing or withdrawing the epidural cannula leaving only the epidural catheter in place.

7. An epidural anaesthesia kit including:
an epidural needle of a gauge of 20 or finer having a lumen having a first axis parallel to said needle that is sized to receive a guide wire therethrough, a tissue piercing point located at a distal end, and a lumen opening disposed at a location at or closely adjacent to said tissue piercing point, the opening being configured to deflect a guide wire exiting from said opening in a direction away from said first axis;
a guide wire sized for use with said epidural needle;
a “loss of resistance” syringe for connection to said needle;
a dilator configured for insertion over said guide wire;
a cannula configured for insertion via said dilator; and
an epidural catheter.

8. The epidural kit of claim 7 wherein the needle gauge is 22 or finer.

9. The epidural kit of claim 7 wherein the “loss of resistance” syringe is an improved sensitivity syringe, said syringe including:
an external generally tubular housing of volume A;
an inner syringe assembly having a barrel of volume B (where A is greater than B) with a plunger rod and piston assembly reciprocally axially movable therein and a discharge opening at its distal end;
said barrel of said internal syringe assembly being located generally co-extensively within said external tubular housing.

10. The epidural kit of claim 9 wherein the syringe further includes indicator means connected with said plunger rod so as to enhance the detection of movement of said internal plunger rod and piston assembly.

11. The epidural kit of claim 9 wherein the external tubular housing of the syringe is sized to be similar to that of a conventional 10 mm syringe and the inner syringe assembly has a significantly smaller volume.

12. The epidural kit of claim 7 to wherein the cannula has a generally straight tubular portion terminating in a discharge section having a discharge opening, said discharge section and discharge opening being configured such that, in use, a catheter passed therethrough is caused to deflect in a direction away from the axial extent of said generally straight tubular portion.

13. The epidural kit of claim 12 wherein the cannula gauge is in the range of 16-20.

14. The epidural kit of claim 12 wherein, in use, the discharged section of the cannula is bent away from the axis of the straight tubular portion.

15. The epidural kit of claim 14 wherein the cannula is formed from a resilient material that has some form of “shape memory” whereby the cannula discharge section can be straightened for insertion through or over a dilator and guide wire and then regain its bent or deflecting configuration when the dilator is removed.

16. The epidural kit of claim 12 wherein the cannula is made from a polymeric material and has a rounded distil end.

17. The epidural kit of claim 12 wherein the cannula includes an integrally formed co-extensive needle guide.

18. An improved epidural needle for anaesthetic via catheter techniques, the needle having:
a lumen having a first axis parallel to said needle that is sized to receive a guide wire therethrough;
a tissue piercing point of pencil point configuration located at a distal end; and
a lumen opening disposed at a location at or closely adjacent said tissue piercing point, said opening being configured to deflect a guide wire exiting from said opening in a direction away from said first axis;
wherein said needle gauge is greater than 21.

19. The improved epidural needle of claim 18 wherein the needle gauge is greater than 22.

20. The improved epidural needle of claim 18 wherein the proximal end of the needle terminates in a connection means for releasable attachment to a “loss of resistance” syringe.

21. A cannula for introducing an epidural catheter, said cannula having a generally straight tubular portion terminating in a discharge section having a discharge opening, said discharge section being made of a resilient material and the discharge opening being configured such that, in use, a catheter passed therethrough is caused to deflect in a direction away from the axial extent of said generally straight tubular portion, the cannula further including a separate integrally formed at least partially co-extensive needle guide.

22. The cannula of claim 21 wherein the gauge is in the range of 16-20.

23. The cannula of claim 21 including connection means at a proximal end for securing the cannula at or adjacent an injection hub forming part of a catheter assembly.

24. The cannula of claim 21 wherein the discharge section is bent in its natural state and the resilient material has some form of shape memory whereby the discharge section can be straightened for insertion through or over a dilator and regain its bent configuration when the dilator is removed.

25. The kit of claim 7 wherein the catheter is a factory preassembled epidural catheter assembly comprising:
an epidural catheter preassembled to a bacterial filter and an injection hub.

26. An improved sensitivity syringe for use in loss of resistance needle placement techniques, said syringe including:
an external generally tubular housing of volume A;
an inner syringe assembly having a barrel of volume B (where A is greater than B) with a plunger rod and piston assembly reciprocally axially movable therein and a discharge opening at its distal end;
said barrel of said internal syringe assembly being located generally co-extensively within said external tubular housing and sized so as to provide a generally tubular space between an inner surface of the external housing and an outer surface of the inner syringe assembly.

27. The improved sensitivity syringe of claim 26 wherein indicator means are disposed within the tubular space and operatively associated with the plunger rod of the inner syringe assembly and the external housing is configured to permit viewing of the indicator means during use of the syringe.

28. The improved sensitivity syringe of claim 26 wherein the ratio between A and B provides a plunger movement magnification of around 300%.

29. The improved sensitivity syringe of claim 26 wherein the external tubular housing is sized to be similar to that of a conventional 10 mm syringe and the internal syringe assembly has a significantly smaller volume.

30. An improved sensitivity syringe for use in loss of resistance needle placement techniques, said syringe including:
a housing sized to be similar to the external dimensions of a conventional 10 ml syringe; and
an internal syringe assembly disposed within the housing, the internal syringe including a barrel having a plunger and piston assembly reciprocally axially movable therein and a discharge opening at its distal end,
wherein the volume of the barrel is significantly less than 10 ml such that movement of the plunger is significantly magnified over that of a plunger in a conventional 10 ml syringe.

31. The improved sensitivity syringe of claim 30 wherein the volume of the barrel is approximately 2 ml.

32. A method of administering epidural anaesthesia according to any one of claims 1 to 6 using a needle according to any one of claims 18 to 20.

33. (canceled)

34. (canceled)

35. A method of administering epidural anaesthesia comprising the steps of:
inserting an epidural needle of a gauge of 20 or finer into the skin over the epidural space, the epidural needle having a lumen having a first axis parallel to said needle that is sized to receive a guide wire therethrough, a tissue piercing point located at a distal end, and a lumen opening disposed at a location at or closely adjacent the tissue piercing point, the opening being configured to deflect a guide wire exiting from the opening in a direction away from said first axis;
using a “loss of resistance” syringe to guide the epidural needle through the external tissue until the tip of the needle including the lumen opening is located within the epidural space;
inserting a guide wire through the needle and into the epidural space;
introducing a cannula that is preloaded onto the outside of the dilator by passing the dilator and cannula assembly over said guide wire until the cannula reaches the noted depth of the epidural space;
removing the dilator and guide wire; and,
inserting the epidural catheter through the cannula and into the epidural space.
